SEQUENCE LISTING (1 sequence)

SEQ ID NO: 1 - protein, 77 aa, human
HPLGSPGSASDLETSGLQEQRNHLQGKLSELQVEQTSLEPLQESPRPTGVWKSREVATEGIRGHRKMVLYTLRAPRS

(19) United States
(12) Patent Application Publication
(10) Pub. No.: US 2013/0137117 A1
(43) Pub. Date: May 30, 2013

(54) METHOD FOR PREPARING PROTEIN IMPRINTED POLYMERS AND USE THEREOF

(71) Applicants: Raphael Levi, Yehud (IL); Ido Margalit, Gan Yavne (IL); Orgad Laub, Tel Aviv (IL); Yarden Dloomy, Modiin (IL)

(72) Inventors: Raphael Levi, Yehud (IL); Ido Margalit, Gan Yavne (IL); Orgad Laub, Tel Aviv (IL); Yarden Dloomy, Modiin (IL)

(21) Appl. No.: 13/386,426

(22) PCT Filed: Jul. 18, 2010
(86) PCT No.: PCT/IL2010/000572

§ 371 Date: May 16, 2012

Related U.S. Application Data
(60) Provisional application No. 61/227,838, filed on Jul. 23, 2009.

(51) Int. Cl. G01N 33/53

(52) U.S. Cl. CPC G01N 33/5308
USPC 435/7.5; 526/318.42; 436/501

(57) ABSTRACT

Methods for preparation of molecularly imprinted polymers and their use for detection of proteins and/or polypeptides in a sample are disclosed. The methods of preparation are based on selecting from available data bases an amino acid sequence of a protein/polypeptide target molecule; cleaving the sequence in-silico with at least one cleaving agent, producing fragments with known composition; selecting at least one such fragment comprising a unique epitope; preparing a synthetic peptide representing the unique epitope; and preparing a molecularly imprinted polymer comprising specific binding sites for the synthetic peptide. For detection of the target protein in a sample, the same cleaving agent used for the in-silico cleavage is used to cleave the target protein to form the specific peptide fragments to which the MIP is specific.

### FIELD OF THE INVENTION

The present invention relates generally to the field of biological analysis, and more particularly to methods for preparation of molecularly imprinted polymers and their use for detection of proteins and/or polypeptides in a sample.

### BACKGROUND OF THE INVENTION

Methods and devices for efficient and accurate detection and quantification of levels of analytes, particularly protein-related analytes, in liquid samples are of particular interest for use in a wide range of applications. Such applications are performed in laboratories, doctors' offices, in the home, in the field; and for analysis of environmental samples include identification or monitoring of physiological or pathological conditions using biological samples; and monitoring of food production lines and environmental samples for the presence of contaminants.

Currently, rapid, real-time, detection and measurement of protein molecules and protein-containing organisms such as bacteria and viruses in liquid samples are performed by sophisticated instruments and by immunoassays that employ target-specific antibodies.

Immunological techniques using labeled reporters have been used widely in biological and medical research and in clinical diagnosis. Despite the abundance of reporters and procedures (Oellerich, 1984, Clin. Chem. Clin. Biochem. 22, 895-904; Gosling, 1990, Clin. Chem. 36, 1408-1427), all the immunodiagnostic techniques predominantly utilize the remarkable affinity and specificity of antibodies.

Antibodies are produced by immunization of animals with the relevant antigen, leading to production of polyclonal antibodies, or by using fused cells, allowing the obtained cell lines to produce monoclonal antibodies.

Antibodies can be raised against most compounds and have been used in various applications (Kohler & Milstein, 1975, Nature 256, 495 497; Oellerich,. 1984, J. Clin. Chem. Clin. Biochem. 22, 895 904 Gosling, 1990. Clin. Chem. 36, 1408 1427; Kurstak, 1986, in Enzyme Immunodiagnosis, Kurstak, ed, pp 5-11, Academic Press, London), ranging from basic research to clinical analysis. However, being bio-macromolecules, antibodies require careful handling and storage, and their production is time-consuming and costly (Kurstak, 1986, in Enzyme Immunodiagnosis, Kurstak, ed, pp 5-11, Academic Press, London), including laborious steps such as conjugation of the hapten to a carrier protein, immunization of animals, bleeding of the animals and isolation and purification of the immunoglobulins.

Attempts to develop alternative ways to obtain antibodies or antibody-like compounds involve use of recombinant techniques applied to bacteria or plants.

Synthetic antibody mimics are a useful alternative. Advantages of such systems include reduction of the need for animal sources, and the possibility of producing synthetic antibodies against molecules for which it is difficult or impossible to raise antibodies, such as immuno-suppressive agents, short peptides or other small molecules. Furthermore, chemically-produced synthetic antibody mimics are more stable than antibodies raised in biological systems, enabling repeated use, performance at higher temperatures, easy sterilization, and greater batch to batch reproducibility.

Thus, there is a need for an immunoassay-like technology in which stable, reproducible and easily prepared, highly selective reagents, such as synthetic polymers, rather than antibodies are used.

One such method involves use of molecularly imprinted polymers (MIPs). Molecular imprinting has attracted much attention in recent years (Alexander et al. 2006, J. Mol. Recognit.; 19: 106-180). A “molecularly imprint polymer” is a polymer which is prepared by polymerizing functional monomers around a template or “print” molecule, which is then removed from the polymer by extraction or other means so that the polymer will selectively absorb the template or print molecule upon re-exposure to the print molecule (Mosbach K. et al., Bio/Technology, 1996, 14, 163-170; Ansell R. J. et al., Curr. Opin. Biotechnol., 1996, 7, 89-94; Wulff G. Angew. Chem. Int. Ed. Engl., 1995, 34, 1812-32; Vidyasankar S. et al., Curr. Opin. Biotechnol., 1995, 6, 218-224; and Shea K. J, Trends In Polymer Science, 1994, 2, 166-173).

Molecularly imprinted polymers demonstrate remarkable recognition properties that have been applied in various fields such as drug separation (Fischer L., et al., J. Am. Chem. Soc., 1991, 113, 9358-9360; Kempe M, et al., J. Chromatogr., 1994, 664, 276-279; Nilsson K., et al., J. Chromatogr., 1994, 680, 57-61), receptor mimics (Ramstrom O., et al., Tetrahedron: Asymmetry, 1994, 5, 649-656; Ramstrom O., et al., J. Mol. Recogn., 1996, 9, 691-696; Andersson L. I., et al., Proc. Natl. Acad. Sci., 1995, 92, 4788-4792; Andersson L. I., Anal. Chem., 1996, 68, 111-117) bio-mimetic sensors (Kriz D., et al., Anal. Chem., 1995, 67, 2142-2144], antibody mimics (Vlatakis G., et al., Nature, 1993, 361, 645-647), template-assisted synthesis (Bystrom S. E., et al, J. Am. Chem. Soc., 1993, 115, 2081-2083) and catalysis (Muller R., et al., Makromol. Chem., 1993, 14, 637-641; Beach J. V., et al., J. Am. Chem. Soc., 1994, Vol. 116, 379-380).

Various methods for imprinting proteins and other macromolecules are known. For example, ionic molecular images of polypeptides have been created by mixing a matrix-material with the intact polypeptide chain to be bound by the molecular image (U.S. Pat. No. 5,756,717). Molecular imprints of cytochrome c, hemoglobin and myoglobin, respectively, have been prepared by polymerizing acrylamide in the presence of each intact protein (U.S. Pat. No. 5,814,223). An imprint of horse myoglobin selectively bound horse myoglobin from a mixture of proteins including whale myoglobin (U.S. Pat. No. 5,814,223).

U.S. Pat. Nos. 5,821,311; 5,872,198, and 5,959,050 to Mosbach, et al. describe certain MIPs, a polymerization process, and symmetrical beads produced by suspension polymerization from functional monomers for use as chromatographic media.

A method for imprinting large biomolecules by the interfacial polymerization of a monomer in the presence of the print molecule and host polymer at the interface between an organic solvent and an aqueous solution is described in U.S. Pat. No. 6,582,971. Imprint compositions that comprise a matrix material defining an imprint of a template molecule, wherein the template molecule typically corresponds to a portion of a macromolecule of interest is disclosed in U.S. Pat. No. 6,979,573. It has been shown that MIPs can be prepared and targeted to Tobacco mosaic virus (Linden et al., 2006, Biomaterials 27, 4165-4168).

Methods that imprint whole proteins are limited due the size of the target to surface binding sites and produce films or gels as the detection agents, a fact that reduces the amount of available binding sites. These entities do not possess high physical, thermal and chemical stability as bulk polymers. In addition, only the surface of the protein is used and inner epitopes that might be of interest are not available for interaction with the MIP.

In the method of “epitope imprinting” (Rachkov and Minoura, 2000, J. Chromatogr A; 889, 111-118), key epitopes are identified on the surface of the protein and the MIP is prepared with a linear peptide representing this epitope as the template.

The key to success in the epitope imprinting of proteins is the identification of the proper epitope to be synthesized as a peptide that will serve as the target in the imprinting process. Currently such epitopes are identified by computerized modeling of crystallographic data and analysis of antibody binding sites, which are not available for many proteins of interest.

In current methods, internal and some external epitopes, which are often conserved and unique, are not available to interact with the MIP.

There is thus a need for molecularly imprinted polymers and methods of use thereof which are devoid of at least some of the limitations of the prior art.

### SUMMARY OF THE INVENTION

The present invention, in at least some embodiments, relates to methods for preparation of molecularly imprinted polymers (MIPs) and their use for detection of proteins and/or polypeptides in a sample.

The method is based on identification of target epitopes from cleavage maps of the protein or polypeptide of interest, preparing synthetic peptides representing these epitopes, and using them as a template for the imprinting process. Detection is performed following cleavage of the sample by an appropriate cleaving agent, and binding of target peptides to the MIP-specific sites.

While reference is generally made to the detection of proteins for illustrative purposes, it is intended to encompass both proteins and polypeptides.

According to some embodiments, there is provided a method for preparation of a molecularly imprinted polymer, the method comprising selecting from available data bases an amino acid sequence of a protein/polypeptide target molecule; in-silico cleaving the amino acid sequence of the target molecule with at least one cleaving agent, producing fragments with known composition; selecting from the fragments at least one comprising a unique epitope; preparing a synthetic peptide representing the unique epitope; and preparing a molecularly imprinted polymer comprising specific binding sites for the synthetic peptide. The protein target molecule may optionally comprises a glycoprotein, lipoprotein or other form of modified protein.

According to some embodiments, the method further comprises exposing the protein target in the liquid sample to at least one cleaving agent producing fragments with known composition; contacting the fragments from the sample with the at least one molecularly imprinted polymer; and detecting binding of the known fragments to the molecularly imprinted polymer.

According to some embodiments, the method further comprises providing a detection device for detecting binding of the known fragments to the molecularly imprinted polymer.

According to some embodiments, the detection device comprises the molecularly imprinted polymer.

According to some embodiments, contacting of the known fragments with said molecularly imprinted polymer is performed prior to introduction of the molecularly imprinted polymer to the device.

According to some embodiments, the method further comprises providing a synthetic peptide-reporter molecule conjugate, wherein the synthetic peptide comprises a unique epitope, such that the synthetic peptide-reporter molecule conjugate competes with the protein target for binding to the molecularly imprinted polymer.

According to some embodiments, the reporter molecule is selected from the group consisting of a chromophore, an enzyme, an affinity-based reporter, 2-(4′-hydroxyphenylazo)benzoic acid, a dye, a fluorescer, a fluorescent dye, a radiolabel, a magnetic particle, a metallic particle, a semiconductor particle, a quantum dot, a colored particle, a fluorescent particle, a metal salt, an enzyme substrate, an enzyme, a chemiluminescer, a photosensitizer and a suspendable particle.

According to some embodiments, the reporter molecule is an affinity-based reporter, and the method further comprising providing a binding pair comprising the affinity-based reporter and a binding element for binding the affinity-based reporter.

According to some embodiments, the binding pair is selected from the group consisting of biotin or a biotin analog or biotin derivative/ biotin binding element; antigen/antibody, hapten/antibody, hormone/receptor, nucleic acid strand/complementary nucleic acid strand, substrate/enzyme, inhibitor/enzyme, protein A or G/immunoglobulin, carbohydrate/lectin, virus/cellular receptor and apoprotein/lipid,cellulose binding protein.

According to some embodiments, the affinity-based reporter comprises biotin and the binding element comprises a biotin binding protein. Alternatively, according to some embodiments, the affinity-based reporter comprises a biotin-binding protein and the binding element comprises biotin.

According to some embodiments, the biotin binding protein is selected from the group consisting of avidin, deglycosylated avidin, and strepavidin,

According to some embodiments, the concentration of reporter molecule is measured by use of an analytical device selected from the group consisting of high pressure liquid chromatograph, gas chromatograph/mass spectrometer, liquid chromatograph/mass spectrometer, ELISA reader, spectrophotometer, absorbance reader, capillary electrophoresis device, fluorescent reader.

According to some embodiments, the concentration of reporter molecule is measured by measurement of an electrical parameter selected from the group consisting of capacitance, resistance, conductance and magnetic parameters.

According to some embodiments, fragments from the test sample are contacted with the molecularly imprinted polymer in a solid phase extraction cartridge.

According to some embodiments, there is provided a device for quantifying a protein target in a liquid sample, the device comprising a molecularly imprinted polymer prepared as described for any of the above embodiments.

According to some embodiments, there is provided a method of detecting at least one analyte in a liquid sample, the method comprising providing an apparatus comprising a molecular imprinted polymer having analyte-specific binding sites; contacting the liquid sample with the molecular imprinted polymer in the apparatus to obtain an effluent comprising unbound analyte; providing a diagnostic device comprising a sample application area for applying the effluent to the device and a detection zone for detecting an amount of unbound analyte present in the effluent.

According to some embodiments, there is provided a method of detecting at least one analyte in a liquid sample, the method comprising providing an analyte specific binding molecule having bound thereto a releasable first binding agent:analyte conjugate and a second binding agent:reporter conjugate binding element having bound thereto a detectable, releasable, second binding agent:reporter conjugate; contacting the analyte specific binding molecule with the liquid sample; and detecting a concentration or presence of second binding agent:reporter conjugate. According to some embodiments, an affinity of the analyte for binding sites of the analyte-specific binding molecule is at least equal to an affinity of the first binding agent:analyte conjugate for analyte-specific binding sites of the analyte-specific binding molecule, such that upon contacting the analyte-specific binding molecule with the analyte in the liquid sample, the analyte is bound and the first binding agent:analyte conjugate is displaced. According to some embodiments, an affinity of the first binding agent:analyte conjugate for analyte-specific binding sites of the reporter conjugate binding element is at least equal to an affinity of the second binding agent:reporter conjugate for binding sites of the second binding agent: reporter conjugate binding element, such that binding of the first binding agent:analyte conjugate displaces second binding agent:reporter conjugate, and displacement of the second binding agent:reporter conjugate is proportional to a concentration of the analyte in the liquid sample.

According to some embodiments, there is provided a method of detecting at least one analyte in a liquid sample, the method comprising providing an analyte-specific binding molecule, a first binding agent:analyte conjugate, a second binding agent:reporter conjugate, and a second binding agent:receptor conjugate binding element; contacting the analyte specific binding molecule with the liquid sample; and detecting a concentration or presence of the second binding agent:reporter conjugate. In some embodiments, the analyte-binding molecule is contacted with the analyte in the liquid sample, such that the analyte and the first binding agent:analyte conjugate analyte compete for analyte-specific binding sites of the analyte-binding molecule, and unbound first binding agent:analyte conjugate flows in a flow path of the liquid sample. In some embodiments, dry second binding agent:reporter conjugate is contacted with unbound first binding agent:analyte conjugate, such that the second binding agent:reporter conjugate and unbound first binding agent:analyte conjugate compete for binding to the reporter-conjugate binding element, and unbound second binding agent:reporter conjugate flows downstream in the flow path of the liquid sample, providing a detectable signal that indicates the concentration of the analyte in the liquid sample.

According to some embodiments, there is provided a method of detecting at least one analyte in a liquid sample, the method comprising providing an analyte-specific binding molecule, and a first binding agent:analyte analog capable of binding to the analyte-binding molecule; providing a second binding agent:reporter conjugate binding element; and contacting the liquid sample with the analyte-binding molecule. In some embodiments, unbound first binding agent:analyte analog is produced by at least one of competition with the analyte for binding sites of the analyte-binding molecule and displacement by the analyte from the analyte-binding molecule, wherein said unbound first binding-agent flows in a flow path of the liquid sample. In some embodiments, an unbound second binding agent:reporter conjugate is produced by at least one of competition with the first binding agent:analyte conjuage and displacement by the first binding agent:analyte conjugate, wherein the presence of unbound second binding agent:reporter conjugate indicates the presence of the analyte in the sample.

As used herein the term “about” refers to ±10%.

### BRIEF DESCRIPTION OF THE DRAWINGS

FIG. 1 is a representation of the rational selection of the epitope peptide that will serve as a template in the preparation of a protein specific MIP; FIG. 2 is an overview of a method of preparation of a protein specific MIP and its use thereof for detection and quantification of protein and polypeptide targets;

FIG. 3 is an overview of an alternative method of preparation of a protein specific MIP and its use thereof for detection and quantification of protein and polypeptide target; and

FIG. 4 is an overview of an alternative method of preparation of a protein specific MIP and its use thereof for detection and quantification of protein and polypeptide target

### DESCRIPTION OF THE PREFERRED EMBODIMENTS

At least some embodiments of the present invention provide methods of making molecularly imprinted polymers, comprising cleaving a target protein with a cleaving agent which produces fragments with known compositions; selecting one or more fragments comprising unique epitopes from the known fragments; preparing synthetic peptides representing the selected epitopes; and preparing a polymer comprising the synthetic peptide.

The present invention, in at least some embodiments, utilizes the vast knowledge of proteomics and the availability of various specific protein cleavage agents. The sequence of all human proteins is currently known, thus, all human proteins can be imprinted using the method disclosed by at least some embodiments of the present invention.

At least some embodiments of the method of the present invention are based upon specific cleavage of a target protein or polypeptide that yields known peptide fragments. The target peptides can then be chosen from the multitude of available peptides according to their adequacy as targets for imprinting, including peptides that originate from internal parts of the protein that are usually not available for interactions.

The present invention, in at least some embodiments, provides a comprehensive method for identification and selection of a unique epitope target of a protein or polypeptide that is used to form a protein-selective polymer.

Pretreatment of the sample with the selected cleaving agent yields known fragments, which do not suffer from conformation restraints. The method of the present invention, in at least some embodiments, thus solves the problem of the need for precise folding of the protein target in order to allow interaction with the protein binding sites of the MIP that were formed against the protein in its three dimensional conformation. In many cases, such as when antibodies are used, precise folding of the protein is needed for the binding agent to recognize its target epitope. Addition of reagents to the sample or different environmental conditions might have an effect on the protein structure and its ability to interact with the detecting agents.

In the method disclosed by at least some embodiments of the present invention, a key feature is the pre-cleavage of the target protein into fragments that are too small to be influenced by the various conditions, and therefore retain their linear composition, making them available for interaction with the diction element.

The peptides that will serve as templates for the imprinting process are selected using available proteomic data that provides the cleavage pattern of the protein or polypeptide of interest. Such data may optionally and preferably be obtained using software tools for predicting protein digestion products, which are readily available and are known to one of ordinary skill in the art. Examples of such tools include, for example, the program ‘PEPTIDE CUTTER’, which is based on protein proteolytic enzyme (such as trypsin or chymotrypsin) or reagent cleavage (such as Lys-C, Arg-C, Asp-N, V8-bicarb, V8-phosp, CN—Br) digests, or combinations thereof. Other examples include tools provided by CLC bio, Denmark (www.cicbio.com).

The final target epitope or epitopes are chosen from the various available epitopes by their uniqueness to the protein or polypeptide analyte and their suitability to the imprinting process.

In some embodiments, selection of the target epitope is based on analyzing specific cleavage maps of the protein target, identifying unique epitope fragments, and performing alignment search using available sequence alignment software tools, such as, for example, Basic Local Alignment Search Tool (BLAST), sequence-context specific BLAST, Position-Specific Iterative (PSI)-BLAST, FASTA, Global:Global (GGSEARCH) or Global:Local (GLSEARCH), HMMER (profile hidden Markov model software), HHpred/HHsearch, inverse document frequency (IDF), Infernal, (Sequenc Alignment/Map) SAM, or SSSEARCH, on the selected candidates to identify fragments that possess unique sequences that are not present in other proteins.

Those epitopes that are determined as being unique to the protein target are then evaluated according to the suitability of their amino acid composition to the imprinting process of choice. Suitability may be based, for example, on factors such as lack of cross-reactivity, and other factors, determination of which is well within the ability of one skilled in the art. In addition, some amino acids are preferred due to their chemical nature. (for example, being negatively or positively charged at neutral pH) that allows better interaction with monomers. Such amino acids include, but are not limited to: Asn, Gln, Asp, Glu, Lys, Arg, His.

The MIPs according to at least some embodiment of the present invention are produced by forming a polymer that contains specific binding sites surrounding the target peptide, using any method known in the art, based on organic and/or inorganic precursors. Suitable imprinting methods include, without limitation, bulk non-covalent imprinting (utilizing non-covalent interactions such as hydrogen bonds, ion-pair interactions, etc.) or covalent imprinting (based on reversible covalent inter-actions) between the print molecule and the functional monomers; precipitation imprinting; semi-covalent imprinting, multi-step swelling polymerization; and sol-gel imprinting.

Briefly, the synthetic template peptide representing the unique epitope is dissolved in a porogen solvent with one or more polymerizable precursors (e.g. monomers, initiators, cross-linkers), and the polymerization is allowed to proceed so as to capture the shape and functional groups of the peptide within the matrix, forming a monolith. Templates interact specifically with functional monomers in different ways to make covalent, non-covalent or semi-covalent complexes. The peptide template molecules are then removed from the imprinted polymer matrix, leaving peptide-specific binding cavities.

According to a preferred embodiment, bulk imprinting is used, which yields highly stable polymers that are porous and have a high degree of binding, since binding is not limited to surface sites only.

The present invention, in at least some embodiments, further provides methods for detection and quantification of a protein target using protein specific MIPs.

In some embodiments, the presence of a protein or polypeptide in a sample is detected by exposing the sample to the same cleavage agent that was used to choose and prepare the synthetic target peptide, such that the protein or polypeptide in the sample is cleaved into defined peptide fragments, including the unique peptide epitope against which the MIP was prepared. The peptides obtained from the sample are then brought into contact with the MIP, such that peptides comprising the unique peptide epitope against which the MIP was prepared are bound by the MIP.

The peptides are not limited only to surface epitopes. Treating the sample with a specific cleavage agent to yield short linear peptides of the entire protein sequence as the targets of the MIP allows utilization of the entire epitope repertoire not being limited to surface epitopes only. The fact that the target peptides are essentially small molecules that can be imprinted within porous polymer allows accessibility of the target molecules to interact with binding sites located inside the porous matrix of the polymer and not be limited to only surface binding sites as is the case when whole proteins are imprinted.

In some embodiments, the unique peptide epitope forms part of an analyte

In some embodiments, the molecularly imprinted polymer is used to detect the presence of an analyte in a liquid sample, by detection of the presence of a unique peptide epitope of the target protein in a liquid sample, based on binding of the peptide epitope by the MIP.

In some embodiments, detection of the protein target further comprises providing a detection device for detecting binding of the peptide epitope by the MIP.

Optionally, the MIP may be provided within the detection device, such that binding of the peptide epitope to the MIP and detection of the binding are carried out within a single device.

Alternatively, binding of the peptide epitope to the MIP may be carried out prior to introduction of the MIP into the detection device, such that binding is carried out externally to the device, and detection of binding is performed within the detection device.

In some embodiments, a synthetic peptide, which is identical to the peptide used for the production of the target-specific MIP, is conjugated to a reporter molecule which provides a detectable signal. The cleaved sample is mixed with a known amount of the peptide-reporter conjugate at a concentration aimed to saturate the peptide-specific binding sites of a precise amount of the peptide-specific MIP, and the mixture is brought into contact with the MIP. Alternatively; the peptide-reporter conjugate can be bound to the MIP prior to the sample application. If present, the peptide in the sample competes with and/or displaces the peptide-reporter-conjugate for the specific binding sites of the MIP. The unbound peptide-reporter conjugate, which after the interaction with the MIP is present in an amount proportional to the initial amount of the protein or polypeptide in the test sample, is used to provide a detectable signal. The reporter may comprise any compound which provides a detectable signal, such as, for example, a chromophore, an enzyme, an affinity-based reporter (such as biotin/avidin), 2-(4′-hydroxyphenylazo)benzoic acid (RABA), dyes, fluorescers, fluorescent dyes, radiolabels, magnetic particles, metallic particles, semiconductor particles, quantum dots, colored particles, fluorescent particles, metal salts, enzyme substrates, enzymes, chemiluminescers, photosensitizers and suspendable particles.

The affinity-based reporter may comprise any molecule or composition capable of recognizing and binding to a specific structural aspect of another molecule or composition.

According to some embodiments, there is further provided a binding pair, comprising the affinity based-reporter and a binding element for binding the affinity-based reporter. In some embodiments, the binding pair comprises biotin (or a biotin analog or biotin derivative)/biotin binding element; antigen/antibody, hapten/antibody, hormone/receptor, nucleic acid strand/complementary nucleic acid strand, substrate/enzyme, inhibitor/enzyme, protein A (or G)/immunoglobulin, carbohydrate/lectin, virus/cellular receptor and apoprotein/lipid,cellulose binding protein. Alternatively, the reporter may be selected from hormones, toxins, lipids, fatty acids, nucleic acids, glycoconjugates, lectins, substrates and ligands, and their respective binding elements.

Generally, either member of the binding pair may be used as the reporter. Hence, for example, the affinity-based reporter may optionally comprise biotin, and the binding element comprises a biotin-binding element, such as, for example, avidin, streptavidin, or a modified form of avidin, such as deglycosylated avidin. Deglycosylated forms of avidin including NeutrAvidin, Extravidin (Sigma-Aldrich), Avidin, native pI neutral-Avip-NNeutral pI, Avidin, deglycosylated pI 10.5-Avip-DG-10.5, and Avidin, deglycosylated pI neutral, Avip-DG-Neutral pI (Affiland, Belgium). Alternatively, the affinity-based reporter may optionally comprise avidin, and the binding element comprises an avidin-binding element, such as biotin.

The signal may, for example, be detectable by ELISA or by a flow-through or lateral flow device such as that described in PCT application no. PCT/IL2008/001688, or by any similar assays that determine and measure the presence of specific reporter molecules. In some embodiments, the binding element is immobilized on a solid support.

In some embodiments, a solution comprising the analyte and analyte-reporter conjugate is contacted with analyte-specific MIP in a preliminary step. In such embodiments, the preliminary step may be performed in an apparatus that allows liquid to pass through the MIP and to be collected. Such an apparatus can be, but is not limited to, a Solid Phase Extraction (SPE) cartridge, wherein the MIP is packed between two frits that hold the MIP in place and allow passing of liquid. When the liquid sample passes through this apparatus the molecules contained within the sample are brought in contact with the MIP and its analyte specific binding sites.

The amount of analyte-reporter conjugate remaining in the solution is proportional to the amount of the analyte in the test sample. The amount of the analyte-reporter conjugate can be determined by a variety of methods, including dedicated lateral flow, flow through and combined devices, such as that of PCT application no. PCT/IL2008/001688, as well as by other methods suitable for the measurement of the reporter molecule.

In some embodiments, wherein the device of PCT application no. PCT/IL2008/001688 is used, the solution comprising the analyte-reporter conjugate is applied to the sample application area, and the amount of reporter conjugate is determined in the detection zone, which is preferably situated downstream of the sample application area.

In a further aspect, there is provided a method for detecting and determining the presence, absence or concentration of a target analyte present in a liquid sample by an assay comprising applying the sample suspected of containing the analyte to an apparatus comprising the MIP. Analyte present in the sample binds to the binding sites of the MIP, displacing analyte analog:reporter conjugate that was pre-bound to said binding sites, which is then collected in the effluent of the apparatus. The effluent is then applied to the sample application area of the single displacement diagnostic device of PCT/IL2008/001688, and flows along or through the solid support, contacting the results zone where it is captured by the analyte analog:reporter conjugate binding element, producing a detectable signal that indicates the presence or amount of the analyte in the sample.

Alternatively, the presence, absence or concentration of a target analyte may be detected using the double displacement device of PCT/IL2008/001688, wherein the analyte is first contacted outside the device with an MIP to which is bound a releasable first binding agent:analyte conjugate. The detection device comprises a sample application area; and a reporter:conjugate binding zone downstream of the sample application area.

Further alternatively, the presence, absence or concentration of a target analyte in a solution may be detected using the single competition device of PCT/IL2008/001688, following a preliminary step of applying the test solution to an apparatus comprising analyte-specific MIP.

Further alternatively, the presence, absence or concentration of a target analyte in a solution may be detected using the double competition device of PCT/IL2008/001688, following a preliminary step of applying the test solution to an apparatus comprising analyte-specific MIP. The molecularly imprinted polymers according to at least some embodiments of the present invention may be used, for example, to detect bio-markers for cardiac-related conditions, specific cancers, infectious diseases, hormonal disorders, etc.

Further alternatively, the presence, absence or concentration of a target analyte in a solution may be detected by measuring the unbound peptide-reporter conjugate remaining in the effluent, using any suitable analytical method or device known in the art for detection of the relevant reporter.

Examples of suitable methods and devices for measurement of the concentration of analyte-reporter conjugate, include, for example high pressure liquid chromatography (HPLC), gas chromatograph/mass spectrometry (GC/MS), liquid chromatography/mass spectrometry (LC/MS), ELISA, spectrophotometry, absorbance reader, capillary electrophoresis, and fluorescent reader.

In some embodiments, the presence, absence or concentration of a target analyte in a solution may be detected by solid phase extraction (SPE), which is an extraction method that uses a solid phase and a liquid phase to isolate one, or one type, of analyte from a solution. The general procedure involves loading a solution onto the SPE phase, washing away undesired components, and then washing off the desired analytes with another solvent into a collection tube. The binding and elution of the target analyte to and from the solid matrix are facilitated by the respective chemical properties of the binding and elution solutions that are prepared specifically for these purposes.

Following exposure to the cleaving agent, the test sample is contacted with the analyte-specific MIP in an SPE cartridge, such that the target fragments bind to the MIP, while the non-relevant molecules are washed away. The specific peptide fragments bound to the MIP in the SPE cartridge are then eluted, and the eluent analyzed by any of the relevant methods for measuring the concentration of the analyte (i.e. HPLC, GC/MS, LC/MS, etc).

According to some embodiments of the present invention, there is further provided a double displacement method for directly detecting and determining the presence, absence or concentration of a target analyte in a liquid sample. The method comprises providing an analyte-binding molecule, having analyte-specific binding sites, to which is bound a releasable first binding agent:analyte conjugate; and a second binding agent:reporter conjugate binding element to which is bound a detectable releasable, second binding agent:reporter conjugate. The liquid sample is contacted with the analyte-binding molecule, such that the first binding agent:analyte conjugate is displaced. Displaced first binding agent:analyte conjugate is then free to contact the second binding agent:reporter conjugate binding element, thereby displacing the second binding agent:reporter conjugate, such that a detectable signal provided by the reporter is detected.

As used herein, the term ‘binding agent:analyte conjugate’ refers to the target analyte, conjugated to a binding agent directly or via a spacer.

The first binding agent:analyte conjugate has high binding affinity to the analyte-specific binding molecule, but lower than the affinity of the unmodified analyte. The first binding agent:analyte conjugate molecules remain attached to the binding sites of the analyte-specific binding molecule even in a moist state unless they are displaced, in a dose-dependent manner, by the target analyte in the tested liquid sample.

The reporter-conjugate binding element has binding sites to which are attached a detectable, releasable, second binding agent:reporter conjugate.

As used herein, the term ‘binding element’ refers to a molecular structure able to specifically bind its respective binding partner with sufficient affinity. Neither the specific sequences nor the specific boundaries of such elements are critical, as long as binding activity is exhibited. Binding characteristics necessarily include a range of affinities, avidities and specificities, and combinations thereof, so long as binding activity is exhibited.

The binding agent:reporter conjugate comprises any reporter entity conjugated to a binding agent capable of specific binding to the same binding element as the binding agent:analyte conjugate.

The second binding agent possesses high affinity to this binding element, but lower than the affinity of the first binding agent:analyte conjugate that is employed in the device. Contact between the first binding agent:analyte conjugate and the binding element therefore causes displacement of the second binding agent:reporter conjugate. Displacement of the second binding agent:reporter conjugate is proportional to a concentration of the analyte in the liquid sample, such that the presence and/or intensity of the detectable signal is related to the amount of analyte in the sample.

As used herein, the term ‘binding partner’ or ‘binding agent’ refers to any molecule or composition capable of recognizing and binding to a specific structural aspect of another molecule or composition. Examples of such binding partners and corresponding molecule or composition include biotin/avidin, antigen/antibody, hapten/antibody, hormone/receptor, nucleic acid strand/complementary nucleic acid strand, substrate/enzyme, inhibitor/enzyme, protein A (or G)/immunoglobulins, carbohydrate/lectin, virus/cellular receptor and apoprotein/lipid.

The releasable first binding agent is selected from the group consisting of biotin, a biotin analog, a biotin derivative, avidin, an avidin derivative, an antigen, Protein A and Protein G, cellulose binding protein, hormones, toxins, lipids, fatty acids, complementary nucleic acid sequences, glycoconjugates, lectins, substrates and ligands.

The releasable second binding agent may be selected from the group consisting of a biotin-binding molecule, an avidin-binding molecule, HABA, DTB, an antigen, Protein A and Protein G, cellulose binding protein, liposomes, hormones, toxins, lipids, fatty acids, complementary nucleic acids, glycoconjugates, lectins, substrates and ligands or their analogs, provided that said second binding agent has lower affinity to the reporter-conjugate binding element than said first binding agent. Biotin analogs are identified in Advances in Protein Chemistry, edited by Anfinsen, Edsall and Richards, Academic Press (1975), pages 104-111.

According to some embodiments of the present invention, there is provided a double competition method for detecting and determining the presence, absence or concentration of a target analyte in a liquid sample. The method, in at least some embodiments, comprises providing an analyte-specific binding molecule, a first binding agent:analyte conjugate, a second binding agent:reporter conjugate, and a second binding agent:receptor conjugate binding element. A sample suspected of containing the analyte is contacted with the analyte-specific binding molecule, so that, if analyte is present in the sample, the first binding agent:analyte conjugate competes with the analyte for the analyte-specific binding sites of the analyte-specific binding molecule. Unbound first binding agent:analyte conjugate then competes with the second binding agent:reporter conjugate for binding to the second binding agent:receptor conjugate binding element.

According to some embodiments of the present invention the method optionally comprise a combination of displacement and competition.

The double displacement/competition approach is designed to facilitate signal formation and interpretation. This approach finds particular utilization with the well-established biotin-avidin system that offers very strong binding affinity together with a multitude of analogs and derivatives that may be used. The double displacement approach is highly applicable when dealing with the “epitope imprinting” method, when the binding agent:analyte conjugate is a synthetic peptide representing an epitope on the target analyte, against which a specific MIP was prepared, conjugated to the binding agent, such as biotin.

This double displacement/competition approach has several advantages over single displacement or competition method when the reporter is directly bound to the target molecule. The first binding agent may be a relatively small molecule, such as biotin, such that adding it to the target molecule does not interfere much with its interaction with the analyte-specific binding molecule. Since the reporter is not bound directly to the analyte, there is great flexibility in the choice of the reporter and the ability to conjugate it to the biotin-derivative. The inability or difficulties in binding a reporter molecule to certain analytes may be a limiting factor for using the displacement approach in these cases. Additionally, it allows increased sensitivity of detection, since various ways of signal amplification known to those skilled in the art may be employed.

Another advantage is that for assays for various analytes the invention employs the same signal formation method, most of the production is similar and only the specific analyte-binding molecule and the analyte conjugated to biotin would need to be developed for every individual product. This makes the time to market of new products shorter and reduces development and production costs.

The principles and operation of the compositions and methods according to the present invention may be better understood with reference to the accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

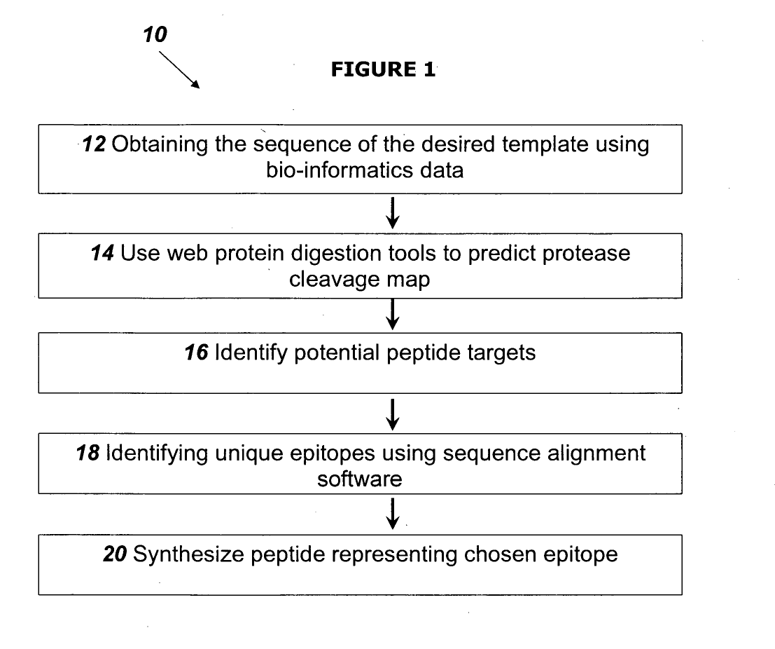

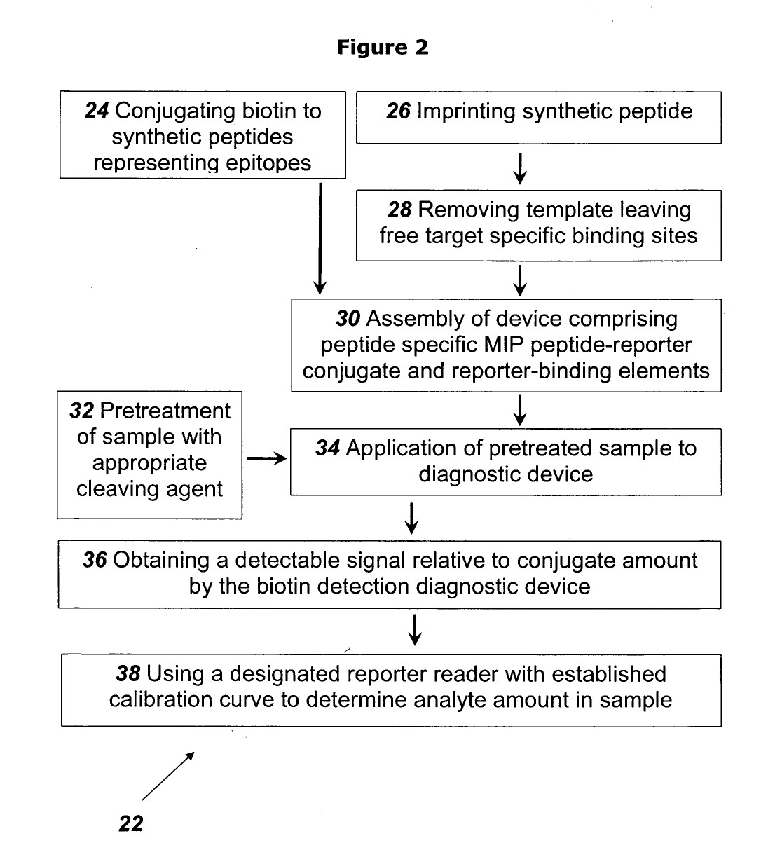

FIG. 1 shows an overview of the method of selection of the epitope peptide according to at least some embodiments of the present invention 10. Step 12 involves obtaining the sequence of the desired target using bio-informatics data. In step 14, software tools for predicting protein digestion products, which are readily available and are known to one of ordinary skill in the art, are used to predict the cleavage map of the target of interest. The cleavage map could be the result of a single agent or a combination of several cleaving agents. In step 16, potential peptide targets are identified according to their suitability for the imprinting process. In step 18, unique epitopes are identified from the potential targets by sequence alignment software tools, as previously described, such as BLAST analysis, which is an algorithm for comparing primary biological sequence information. Step 20 involves synthesis of the peptide representing the selected epitope.

FIG. 2 shows an overview of the process 22 of preparing a protein-specific MIP and its use for detection and quantification of protein and polypeptide targets. Process 22 comprises step 24, wherein a reporter molecule, such as biotin, is conjugated to synthetic peptides representing unique epitopes identified in step 18 of FIG. 1. Step 26 comprises imprinting a synthetic peptide corresponding to the synthetic peptides representing unique epitopes identified in step 18 of. FIG. 1. The imprinted synthetic peptide obtained in step 26 is treated in step 28 to remove the template, leaving free target-specific binding sites.

In step 30, the products of steps 24, 26 and 28, are introduced into a device comprising peptide-specific MIP, peptide-reporter conjugate, and reporter-binding elements. Step 32 involves pretreatment of the sample to be tested with the appropriate cleaving agent. Step 34 comprises application of the treated sample from step 32 to the diagnostic device constructed at step 30. In step 36 the amount of free peptide-conjugate yields a detectable signal by the reporter, on the device constructed in step 30. A calibration curve is constructed for a designated reporter reader in step 38, and used to quantify the amount of analyte in the sample.

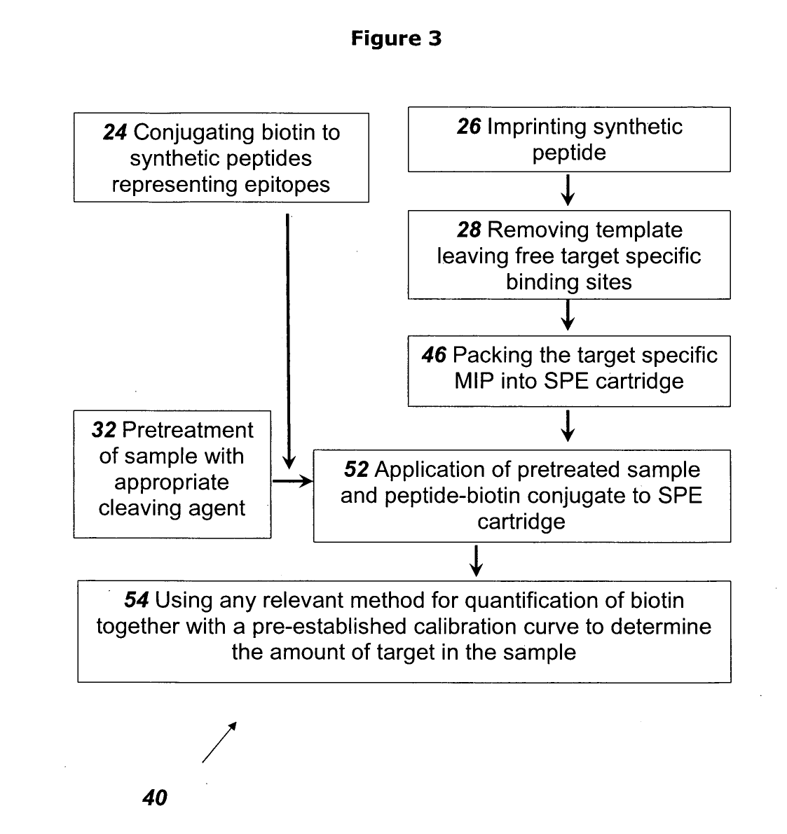

FIG. 3 shows an overview of the process 40 of preparing a protein-specific MIP and its use for detection and quantification of protein and polypeptide targets. Process 40 comprises step 24, wherein a reporter molecule, such as biotin, is conjugated to synthetic peptides representing unique epitopes identified in step 18 of FIG. 1. Step 26 comprises imprinting a synthetic peptide corresponding to the synthetic peptides representing unique epitopes identified in step 18 of FIG. 1. The imprinted synthetic peptide obtained in step 26 is treated in step 28 to remove the template, leaving free target-specific binding sites.

In step 46, the target specific MIP is packed into a solid phase extraction cartridge by placing a frit with pore size smaller than the size of the MIP particles at the bottom of the cartridge, applying the MIP particles on top the fit place another fit on top of the MIP and press them all together to fix them in place.

Step 32 involves pretreatment of the sample to be tested with the appropriate cleaving agent. Step 52 comprises application of the treated sample from step 32 and the peptide biotin conjugate from step 24 to the SPE cartridge packed with MIP of step 46. In step 54 the amount of biotin (which corresponds to the amount of the excess conjugated peptide, which is proportional to the amount of the peptide target in the sample) is quantified using any suitable method known in the art. A calibration curve is constructed for the biotin, and used to quantify the amount of analyte in the sample.

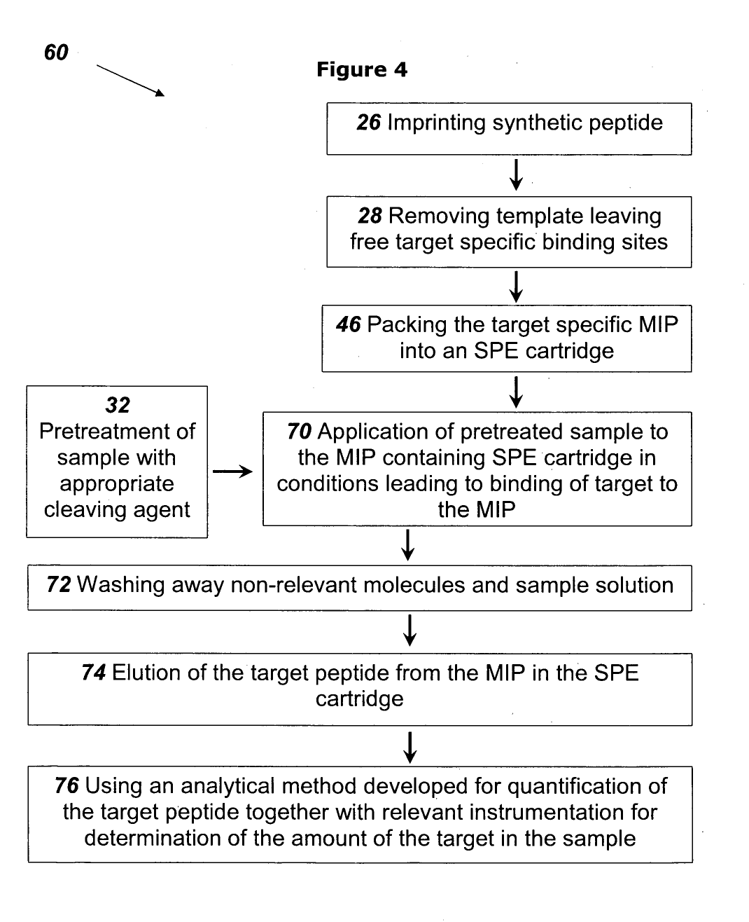

FIG. 4 shows an overview of the process 60 of preparing a protein-specific MIP and its use for detection and quantification of protein and polypeptide targets. Step 26 comprises imprinting a synthetic peptide corresponding to the synthetic peptides representing unique epitopes identified in step 18 of FIG. 1. The imprinted synthetic peptide obtained in step 26 is treated in step 28 to remove the template, leaving free target-specific binding sites.

In step 46, the target specific MIP is packed into a solid phase extraction cartridge. Step 32 involves pretreatment of the sample to be tested with the appropriate cleaving agent.

Step 70 comprises application of the treated sample from step 32 to the SPE cartridge packed with MIP of step 46. In step 72, non-relevant molecules and sample solution are washed away. The target peptide from the MIP in the SPE cartridge is then released from the MIP and eluted in step 74. In step 76, any suitable analytical method developed for quantification of the target peptide is used, together with relevant instrumentation, to determine the amount of the target in the sample.

### EXAMPLES

Reference is now made to the following examples, which together with the above descriptions, illustrate the invention in a non limiting fashion.

### Example 1

This example describes the identification of an epitope on the NT-proBNP protein that is suitable for us as a template for a NT-proBNP specific MIP, and the preparation of a synthetic peptide representing this epitope, as well as a biotin-peptide conjugate.

The sequence of the NT-proBNP, as obtained from the web site of the National Center for Biotechnology Information (NCBI) is as follows:

10 20 30 40
HPLGSPGSAS DLETSGLQEQ RNHLQGKLSE LQVEQTSLEP
50 60 70
LQESPRPTGV WKSREVATEG IRGHRKMVLY TLRAPRS

The on-line program “PEPTIDE CUTTER” was used to obtain the cleavage map of the NT-proBNP. This is a web based program that searches a protein sequence from the SWISS-PROT and/or TrEMBL databases or a user-entered protein sequence for protease cleavage sites. Single proteases and chemicals, a selection or the whole list of proteases and chemicals can be used. Different forms of output of the results are available: Tables of cleavage sites are either grouped alphabetically according to enzyme names or sequentially according to the amino acid number. A third option for output is a map of cleavage sites. The sequence and the cleavage sites mapped onto it are grouped in blocks, the size of which can be chosen by the user. This helps the user to find a convenient form of print-out.

Several fragments having size between 8 to 13 amino acids were identified. Their sequences were analyzed by the on-line BLAST program to look for the presence of these sequences in other human proteins. A fragment of 10 amino acids (Gln-Glu-Ser-Pro-Arg-Pro-Thr-Gly-Val-Trp) that is produced by cleavage with Chymotrypsin was chosen as the preferred target. A peptide representing this epitope, and a biotin-peptide conjugate of the same peptide were provided by a commercial source (GL Biochem, Shanghai Ltd., China).

### Example 2

This example describes the preparation of NT-proBNP specific MIP.

Preparation was in accordance with the methods set forth in the review by Yan and Row (Int. J. Mol. Sci. 2006, 7, 155-178) as follows. The functional monomer, methacrylic acid (MAA) (Cat. No. 155721, Aldrich) was mixed with the target print molecule, in this case the synthetic peptide described in Example 1 above, together with the cross-linking monomer ethylene glycol dimethacrylate (EGDMA), (Cat. No. 33568-1, Aldrich), in 3% water in acetonitrile (Cat. No. 360457,Sigma-Aldrich), together with the initiator 2,2′-azobis(2,4-dimethylvaleronitrile) (Cat. No. 002094, Chemos GmBH, Germany). The mixture was degassed and purged with nitrogen for 5 min and the polymerization took place for 16 hours at 40° C., resulting in a rigid insoluble polymer with NT-proBNP -specific binding cavities present within the polymeric network. The bulk polymer was ground and wet sieved in ethanol through 63 and 25 μm sieves. The fraction of particles between 25 to 63 μm was collected for further use. The print molecule was extracted by extensive washing of the particles with methanol-acetic acid (9/1, v/v). The polymer particles were dried under vacuum and stored desiccated.

### Example 3

This example describes the use of the NT-proBNP-specific MIP of Example 2 in the detection of NT-proBNP in a sample, using a rapid lateral flow and/or flow through MIP based device as disclosed in patent application No. PCT/IL2008/001688. The device is assembled using the NT-proBNP specific MIP as the detection element.

The sample containing the NT-proBNP is digested with Chymotrypsin under conditions that ensure full cleavage of the proteins in the sample. One unit of enzyme hydrolyzes 1.0 μmole of the target protein per minute at pH 7.8 at 25° C. The time needed for full cleavage is calculated according to the amount of target protein and the amount of enzyme in the reaction mixture.

Upon the treatment of the sample with Chymotrypsin, all the proteins in the sample are cleaved according to their respective cleavage sites, producing fragments which include the peptide corresponding to the epitope peptide that was used to prepare the NT-proBNP specific MIP, in an amount proportional to the initial amount of the NT-proBNP polypeptide in the sample. The pretreated sample is then applied to the device described in Example 2, that contains, beside the NT-proBNP specific MIP, also the biotin-peptide conjugate disclosed in Example 1, in an amount that is designed to be captured in its entirety by the NT-proBNP specific MIP on the device in the absence of the corresponding target fragment. The target fragment from the sample competes with the biotin-peptide conjugate for the specific binding sites of the NT-proBNP specific MIP on the device. An amount of biotinilated peptide is left unbound in an amount proportional to the amount of the NT-proBNP in the sample. The molecules of the unbound biotinilated peptide provide o a readable signal as described in patent application No. PCT/IL2008/001688.

It is envisioned that in certain cases a mixture of MIPs prepared each against a different peptide fragment might be used as the detection element, each interacting with its specific peptide and its respective biotin conjugate and all contributing to the final result signal.

### Example 4

This example describes the detection and quantification of NT-proBNP in a sample using a biotin microplate determination method.

Wells of a high protein binding microplate are coated with avidin or other biotin-binding protein and blocked by bovine serum albumin (BSA) to eliminate residual binding sites on the wells. The biotin-containing sample is then incubated in the wells, where any biotin-containing molecules bind to the biotin-binding protein coating the well.

After washing, a solution containing horseradish peroxidase (HRP) conjugated to biotin is incubated in the wells where it binds to available binding sites of the biotin binding protein.

After another washing step, HRP substrate 3,3,5,5-tetramethylbenzidine (TMB) is added to the wells.

After incubation for 30-60 minutes (according to the color development of the calibration curve wells) the reaction is stopped with H₂SO₄ and the resulting color is read in a plate reader at 450 nm. Higher amounts of biotin-containing molecules in the sample lead to blocking of more biotin binding sites of the protein coating the well, leaving fewer sites for the biotinilated HRP enzyme, and eventually to reduction of the color signal in comparison to the control wells which contain only buffer with no biotin. Incubation of known amounts of biotinilated molecules allows the establishment of a calibration curve that can be used to quantify the amount of biotinilated molecule in the unknown samples.

5 mg of NT-proBNP specific MIP are incubated in filtration tubes (with 0.45-1 μm filter) together with a sample pretreated as described in Example 3 and biotin-peptide conjugate as disclosed in Example 1, in an amount that is designed to be captured in its entirety by the NT-proBNP specific MIP in the absence of the corresponding target fragment. The NT-proBNP specific fragment from the sample competes with the biotinilated peptide for the specific binding sites of the MIP. An amount of unbound biotinilated peptide is left unbound in an amount proportional to the amount of the NT-proBNP in the sample. The tubes are then centrifuged and the liquid sample containing the unbound biotinilated peptide is then analyzed by the biotin microplate determination method described above.

### Example 5

The previous examples describe methods involving competition of the biotinilated peptide and the specific peptide fragment from the sample for binding sites of the specific MIP. The same procedures can be performed with MIP pre-loaded with biotinilated peptide that is displaced by the specific fragment from the sample, wherein this fragment possess higher affinity to the MIP binding sites than the biotinilated peptide. The analyte conjugate possesses the same functional groups enabling binding to the analyte specific MIPs but since the MIP is analyte specific the addition of biotin to the analyte conjugate reduces its binding affinity compared to the target analyte due to the difference in the overall structure.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method for preparation of a molecularly imprinted polymer, the method comprising:
(i) Selecting from available data bases an amino acid sequence of a protein/polypeptide target molecule;
(ii) In-silico cleaving said amino acid sequence of said target molecule with at least one cleaving agent, producing fragments with known composition
(iii) selecting from said fragments at least one comprising a unique epitope;
(iv) preparing a synthetic peptide representing said unique epitope; and
(v) preparing a molecularly imprinted polymer comprising specific binding sites for said synthetic peptide.

2. The method of claim 1, further comprising quantifying a protein target in a liquid sample by
(vi) exposing said protein target in said liquid sample to said at least one cleaving agent producing said fragments with known composition;
(vii) contacting said fragments from said sample with said at least one molecularly imprinted polymer; and
(viii) detecting binding of said known fragments to said molecularly imprinted polymer.

3. The method of claim 1, further comprising providing a detection device for detecting binding of said known fragments to said molecularly imprinted polymer.

4. The method of claim 3, wherein said detection device comprises said molecularly imprinted polymer.

5. The method of claim 3, wherein said contacting of said known fragments with said molecularly imprinted polymer is performed prior to introduction of said molecularly imprinted polymer to said device.

6. The method of any of claim 5, further comprising providing a synthetic peptide-reporter molecule conjugate, wherein said synthetic peptide comprises said unique epitope, such that said synthetic peptide-reporter molecule conjugate competes with said protein target for binding to said molecularly imprinted polymer.

7. The method of claim 6, wherein said reporter molecule is selected from the group consisting of a chromophore, an enzyme, an affinity-based reporter, 2-(4′-hydroxyphenylazo) benzoic acid, a dye, a fluorescer, a fluorescent dye, a radiolabel, a magnetic particle, a metallic particle, a semiconductor particle, a quantum dot, a colored particle, a fluorescent particle, a metal salt, an enzyme substrate, an enzyme, a chemiluminescer, a photosensitizer and a suspendable particle.

8. The method of claim 7, wherein said reporter molecule is an affinity-based reporter, the method further comprising providing a binding pair comprising said affinity-based reporter and a binding element for binding said affinity-based reporter.

9. The method of claim 8, wherein said binding pair is selected from the group consisting of biotin or a biotin analog or biotin derivative/biotin binding element; antigen/antibody, hapten/antibody, hormone/receptor, nucleic acid strand/complementary nucleic acid strand, substrate/enzyme, inhibitor/enzyme, protein A or G/immunoglobulin, carbohydrate/lectin, virus/cellular receptor and apoprotein/lipid,cellulose binding protein.

10. The method of claim 9, wherein said affinity-based reporter comprises biotin and said binding element comprises a biotin binding protein.

11. The method of claim 9, wherein said affinity-based reporter comprises a biotin-binding protein and said binding element comprises biotin.

12. The method of any of claim 11, wherein said biotin binding protein is selected from the group consisting of avidin, deglycosylated avidin, and strepavidin,

13. The method of claim 12, wherein the concentration of said reporter molecule is measured by use of an analytical device selected from the group consisting of high pressure liquid chromatograph, gas chromatograph/mass spectrometer, liquid chromatograph/mass spectrometer, ELISA reader, spectrophotometer, absorbance reader, capillary electrophoresis device, fluorescent reader.

14. The method of claim 13, wherein the concentration of said reporter molecule is measured by measurement of an electrical parameter selected from the group consisting of capacitance, resistance, conductance and magnetic parameters.

15. The method of claim 2, wherein said fragments from said test sample are contacted with said molecularly imprinted polymer in a solid phase extraction cartridge.

16. A device for quantifying a protein target in a liquid sample, the device comprising a molecularly imprinted polymer prepared according to claim 1.

17. A method of detecting at least one analyte in a liquid sample, the method comprising:
a) providing an apparatus comprising a molecular imprinted polymer having analyte-specific binding sites;
b) contacting said liquid sample with said molecular imprinted polymer in said apparatus to obtain an effluent comprising unbound analyte;
c) providing a diagnostic device comprising a sample application area for applying said effluent to said device and a detection zone for detecting an amount of said unbound analyte present in said effluent.

18. A method of detecting at least one analyte in a liquid sample, the method comprising
a) providing an analyte specific binding molecule having bound thereto a releasable first binding agent:analyte conjugate and a second binding agent:reporter conjugate binding element having bound thereto a detectable, releasable, second binding agent:reporter conjugate;
b) contacting said analyte specific binding molecule with the liquid sample; and
c) detecting a concentration or presence of said second binding agent:reporter conjugate,
wherein an affinity of the analyte for said binding sites of said analyte-specific binding molecule is at least equal to an affinity of said first binding agent: analyte conjugate for said analyte-specific binding sites of analyte-specific binding molecule,
wherein upon contacting said analyte-specific binding molecule with the analyte in the liquid sample, the analyte is bound and said first binding agent:analyte conjugate is displaced,
wherein an affinity of said first binding agent:analyte conjugate for said analyte-specific binding sites of said reporter conjugate binding element is at least equal to an affinity of said second binding agent:reporter conjugate for said binding sites of said second binding agent: reporter conjugate binding element, wherein binding of said first binding agent:analyte conjugate displaces second binding agent:reporter conjugate, and wherein displacement of said second binding agent:reporter conjugate is proportional to a concentration of the analyte in the liquid sample.

19. A method of detecting at least one analyte in a liquid sample, the method comprising:
a) providing an analyte-specific binding molecule, a first binding agent: analyte conjugate, a second binding agent:reporter conjugate, and a second binding agent:receptor conjugate binding element;
b) contacting said analyte specific binding molecule with the liquid sample; and
c) detecting a concentration or presence of said second binding agent:reporter conjugate
wherein upon contacting said analyte-binding molecule with the analyte in the liquid sample, the analyte and said first binding agent:analyte conjugate analyte compete for said analyte-specific binding sites of said analyte-binding molecule, and
wherein said unbound first binding agent:analyte conjugate flows in a flow path of the liquid sample,
wherein upon contacting said dry second binding agent:reporter conjugate with said unbound first binding agent:analyte conjugate, said second binding agent:reporter conjugate and said unbound first binding agent:analyte conjugate compete for binding to said reporter-conjugate binding element,
wherein said unbound second binding agent:reporter conjugate flows downstream in said flow path of the liquid sample, providing a detectable signal that indicates the concentration of the analyte in the liquid sample.

20. A method of detecting at least one analyte in a liquid sample, the method comprising
providing an analyte-specific binding molecule, and a first binding agent:analyte analog capable of binding to said analyte-binding molecule;
providing a second binding agent:reporter conjugate binding element; and
contacting said liquid sample with said analyte-binding molecule, wherein unbound first binding agent:analyte analog is produced by at least one of competition with the analyte for binding sites of the analyte-binding molecule and displacement by said analyte from said analyte-binding molecule,
wherein said unbound first binding-agent flows in a flow path of the liquid sample,
wherein an unbound second binding agent:reporter conjugate is produced by at least one of competition with said first binding agent:analyte conjuage and displacement by said first binding agent:analyte conjugate,
wherein the presence of unbound second binding agent:reporter conjugate indicates the presence of the analyte in the sample.
